(19) United States
(12) Patent Application Publication
(10) Pub. No.: US 2013/0131500 A1
(43) Pub. Date: May 23, 2013

(54) IN-SITU PROXIMITY RECOGNITION APPARATUS

(71) Applicant: Covidien LP, Mansfield, MA (US)

(72) Inventor: Alexey Sharonov, Bethany, CT (US)

(73) Assignee: Covidien LP, Mansfield, MA (US)

(21) Appl. No.: 13/647,484

(22) Filed: Oct. 9, 2012

Related U.S. Application Data
(60) Provisional application No. 61/561,482, filed on Nov. 18, 2011.

(51) Int. Cl. A61B 6/12

(52) U.S. Cl. 600/424

(57) ABSTRACT

The present disclosure is directed to a medical device comprising an elongate member having a proximal portion and a distal portion, at least one adapter operably coupled to the elongate member, said adapter including at least one inductor configured to convert electromagnetic energy into an electrical current, and at least one indicator operably coupled to said inductor.

### CROSS REFERENCE TO RELATED APPLICATION

The present application claims the benefit of and priority to U.S. Provisional Application Ser. No. 61/561,482, filed on Nov. 18, 2011, the entire contents of which are incorporated herein by reference.

### BACKGROUND

1. Technical Field

The present disclosure relates to the use of medical instruments. More particularly, the present disclosure is directed to estimating the proximity of a medical device to a target location.

2. Background of the Related Art

During minimal invasive surgery, it is often important to know where a surgical tool or internal features are located. Surgeons can have a difficult time locating a desired location inside a patient to accurately operate on or position an instrument in the patient. Devices exist that can help in situ location of instruments, but they require emitters to be present on the medical device itself. This requires the use of potentially toxic batteries or powerful magnets for the medical device to generate a signal that can be read. A way to indicate position with respect to a desired location inside a patient without the use of emitter on the medical device would lead to a safer more cost effective device.

### SUMMARY

In accordance with at least one aspect of the present disclosure, a medical device includes an elongate member having a proximal portion, a distal portion, and at least one adapter operably coupled to the elongate member. The adapter includes at least one inductor configured to convert electromagnetic energy into an electrical current. The medical device and/or adapter may include at least one indicator operably coupled to the inductor.

In accordance with at least one aspect of the present disclosure, the at least one adapter may be connected integrally upon the distal portion. In another aspect, the at least one adapter may also be removably connected to the distal portion.

In accordance with at least one aspect of the present disclosure, the at least one indicator may be removably connected to the distal portion. In another aspect, the at least one indicator may be connected to the proximal portion.

In accordance with at least one aspect of the present disclosure, the at least one indicator may be integrally connected to the elongate member.

In accordance with at least one aspect of the present disclosure, the at least one indicator may be at least one visual indicator configured to convert the electrical current into light. The at least one visual indicator may be at least one light-emitting diode (LED). The indicator may be at least one LED connected to the distal portion.

In accordance with at least one aspect of the present disclosure, the at least one indicator may be at least one audible indicator configured to convert the electrical current into audible sound.

In accordance with at least one aspect of the present disclosure, the adapter and indicator may be configured to selectively secure to the elongate member and to be selectively removable therefrom.

In accordance with at least one aspect of the present disclosure, a proximity sensing system for determining proximity to a desired location inside a patient includes at least one medical device having an elongate member including a distal portion and a proximal portion, at least one power supply, at least one coil operably coupled to the at least one power supply and configured to selectively output electromagnetic energy, at least one adapter comprising at least one inductor operably coupled to the distal portion and configured to inductively couple to the coil and convert electromagnetic energy into an electrical current, and at least one indicator operably coupled to the inductor.

In accordance with at least one aspect of the present disclosure, the indicator may be at least one LED. The indicator may be an LED that is connected to the distal portion.

In accordance with at least one aspect of the present disclosure, a method of determining the proximity of a medical device to a desired location inside a patient, includes providing at least one power supply, at least one coil operably coupled to the at least one power supply and configured to selectively output electromagnetic energy, at least one medical device having a elongate member comprising a distal portion, a proximal portion and at least one adapter including at least one inductor operably coupled to the distal portion and configured to inductively couple to the at least one coil and convert electromagnetic energy into electrical current, and at least one indicator operably connected to said inductor.

In accordance with at least one aspect of the present disclosure, the method further comprises placing the at least one coil outside of the patient over the desired location, allowing electrical current to pass through the at least one coil to create electromagnetic energy, positioning the distal portion of the medical device inside the patient, receiving electromagnetic energy from the coil using the inductor thereby inductively coupling the inductor to the coil to create electrical current in the inductor, and powering the indicator using electrical current from the inductor.

In accordance with at least one aspect of the present disclosure, the method may further include determining an intensity of the indicator when the medical device is in a first position relative to the coil and a second position relative to the coil, wherein the intensity is proportional with respect to the relative positioning of the coil and the medical device.

In accordance with at least one aspect of the present disclosure, the method may further include comparing the intensity at the first position with the intensity at the second position to determine if the medical device has moved closer or further from the desired location.

In accordance with at least one aspect of the present disclosure, the indicator may be at least one LED that is connected to the distal portion.

In accordance with at least one aspect of the present disclosure, the at least one LED may comprise at least one color. In accordance with at least one aspect of the present disclosure, the at least one LED comprises a first color in a first condition and a second color at a second condition.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1A is a perspective view of an embodiment of medical device having an elongate member in accordance with this disclosure;

FIG. 1B is a perspective view of a distal portion of the elongate member of FIG. 1A, in accordance with this disclosure;

FIG. 2 is a perspective view of an embodiment of an elongate member in accordance with this disclosure;

FIG. 3 is a view of a system in accordance with this disclosure; and

FIG. 4 is a circuit diagram of an inductor operably coupled to a diode in accordance with this disclosure.

### DETAILED DESCRIPTION

Particular embodiments of the present disclosure are described hereinbelow with reference to the accompanying drawings; however, the disclosed embodiments are merely examples of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Like reference numerals may refer to similar or identical elements throughout the description of the figures. As shown in the drawings and described throughout the following description, as is traditional when referring to relative positioning on a surgical instrument, the term “proximal” refers to the end of the apparatus that is closer to the user and the term “distal” refers to the end of the apparatus that is farther away from the user. The term “clinician” refers to any medical professional (e.g., doctor, surgeon, nurse, or the like) performing a medical procedure involving the use of embodiments described herein.

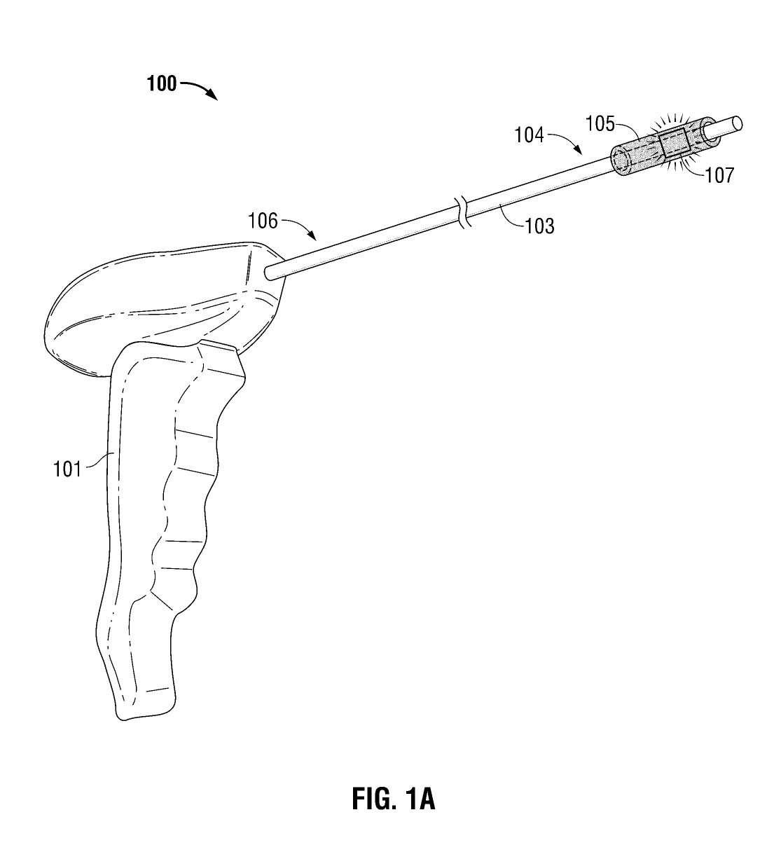

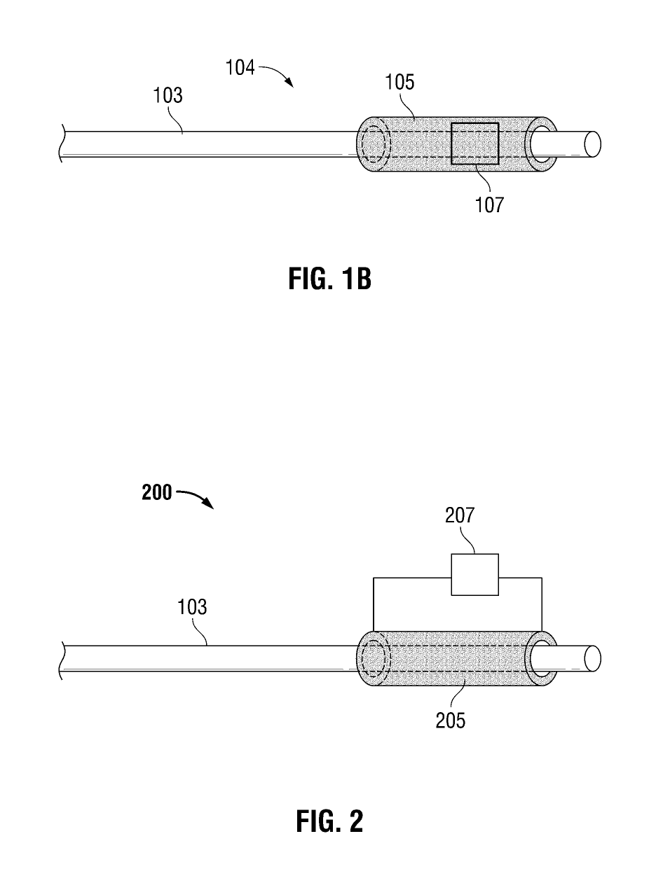

In accordance with at least one aspect of the present disclosure, a medical device is, described herein. With reference to FIGS. 1A and 1B, the medical device 100 may have an elongate member 103 including a proximal portion 106 and a distal portion 104. The elongate member 103 may be integrally or removably coupled to a housing 101. Housing 101 may be operably coupled to an end effector disposed on the elongate member 103. Examples of end effectors include but are not limited to vessel sealing devices, linear stapling devices, circular stapling devices, cutters, etc. Furthermore, an end effector may be configured to deploy a probe, implant, or other device in a desired condition.

At least one adapter 105 is operably coupled to the elongate member 103. The operable coupling between adapter 105 and elongate member 103 may be any suitable integral or removable connection, such as but not limited to a latch, an adhesive, and a contact friction connection.

The adapter 105 includes at least one inductor configured to convert electromagnetic energy into an electrical current. The at least one inductor may be any device suitable for converting electromagnetic (EM) energy into electrical current, such as but not limited to at least one wire coil, at least one antenna configured to accept at least one desired EM frequency, and combinations thereof. The inductor is configured to inductively couple and accept EM energy from an emitter, thereby creating an electrical current having an intensity proportional to the distance from the emitter. For example, the intensity may increase as distance from the emitter decreases.

Adapter 105 may simply be a wire coil inductor that is either integrally or removably connected to the elongate member 103. The at least one adapter 105 may be connected integrally upon the distal portion 104 such that it forms at least a portion of the elongate member 103. In some embodiments, the at least one adapter 105 is removably connected to the distal portion 104 such that the adapter 105 may be replaced as desired. For example, the adapter 105 may be able to slide on to the elongate member 103 and selectively secure to the elongate member 103, and then further be releasable such that adapter 105 may slide off of the elongate member 103.

The medical device 100 and/or adapter 105 may include at least one indicator 107 operably coupled to the inductor in adapter 105 such that inductor 105 and indicator 107 are in a permanent or selectable electrical connection. As shown in FIG. 1B, indicator 107 may be disposed integrally or removably upon the adapter 105. However, as shown in FIG. 2, indicator 207 may be disposed elsewhere on or near the medical device 200 as long as indicator 207 maintains an operable electrical connection with adapter 205. For example, referring back to FIGS. 1A and 1B, the at least one indicator 107 may be integrally or removably connected to the distal portion 104. In other embodiments, the at least one indicator 105 is connected to the proximal portion 106 of elongate member 103.

In some embodiments, the at least one indicator 107 is integrally connected to the elongate member 103. However, the indicator 107 may also be removably connected to the elongate member 103 so as to allow removal or replacement of indicator 107. In some embodiments, both the adapter 105 and indicator 107 may be configured to selectively secure to the elongate member 103 and to be selectively removable therefrom.

The at least one indicator 107 may be at least one visual indicator configured to convert the electrical current produced by the inductor(s) in adapter 105 into visible or invisible light. The light produced may be of any desired wavelength range including but not limited to visible light, RF, microwave, ultraviolet, and infrared. For example, where the indicator 107 emits a visible light, the user may look directly at the light source to determine intensity. Where the indicator 107 emits an invisible frequency of light, the user may use a detector to sense the invisible light emission and intensity thereof.

In at least some embodiments, the at least one visual indicator is at least one light-emitting diode (LED). The at least one LED may be of any color, size, or shape desired. In some embodiments, the at least one LED has a first color in a first condition and a second color at a second condition.

In some embodiments, multiple LED's may be present having the same or a combination of colors, shapes, and sizes. For example, a first LED of a first color may operate a first range of voltages/currents and a second LED of a second color may operate at second range of voltages/currents. Such color cascading may be extrapolated to any number of LED's and current ranges. Referring to FIG. 4 which shows a circuit diagram of an adapter-indicator assembly 400, inductor 403 is connected to diode 405. At least one capacitor 407 may also be disposed in parallel with the inductor 403 and the diode 405.

Referring back to FIGS. 1A and 1B, using a visual indicator allows the medical device 100 to produce light of increasing intensity as the adapter 105 is moved closer to an emitter because the inductor produces increasing current with decreasing distance to the emitter. Thus, the user may approximate the position of the medical device 100 relative to the emitter by viewing or measuring the intensity of the light. Furthermore, in embodiments where a plurality of colored light sources exists, the user may approximate the position of the medical device 100 relative to the emitter by viewing a change in the color of the activated light sources.

In some embodiments, the light may also be pulsed relative to the amount of current being produced by the inductor. For example, indicator 105 may comprise a light configured to pulse with increasing frequency proportional to the inductively produced current such that the closer the adapter 105 is positioned to a desired location, the more frequently the light blinks. Thus, the user may approximate the position of the medical device 100 relative to the emitter by viewing or measuring the blink frequency of the light.

In some embodiments, the at least one indicator 107 is at least one audible indicator configured to convert the electrical current into audible sound such as at least one speaker. The sound created may be of any desired frequency. Using an audible indicator allows the medical device 100 to produce sound of increasing intensity as the adapter 105 is moved closer to an emitter because the inductor produces increasing current with decreasing distance to the emitter. Thus, the user may approximate the position of the medical device 100 relative to the emitter by listening to or measuring the intensity of the sound. As described above for visible indicators, the audible indicator may also be pulsed at a constant intensity based on the relative distance to the emitter. For example, the audible indicator may beep at an increasing frequency as the adapter 105 approaches the emitter.

The at least one indicator 107 may comprise any combination of audible and visual indicators as described above.

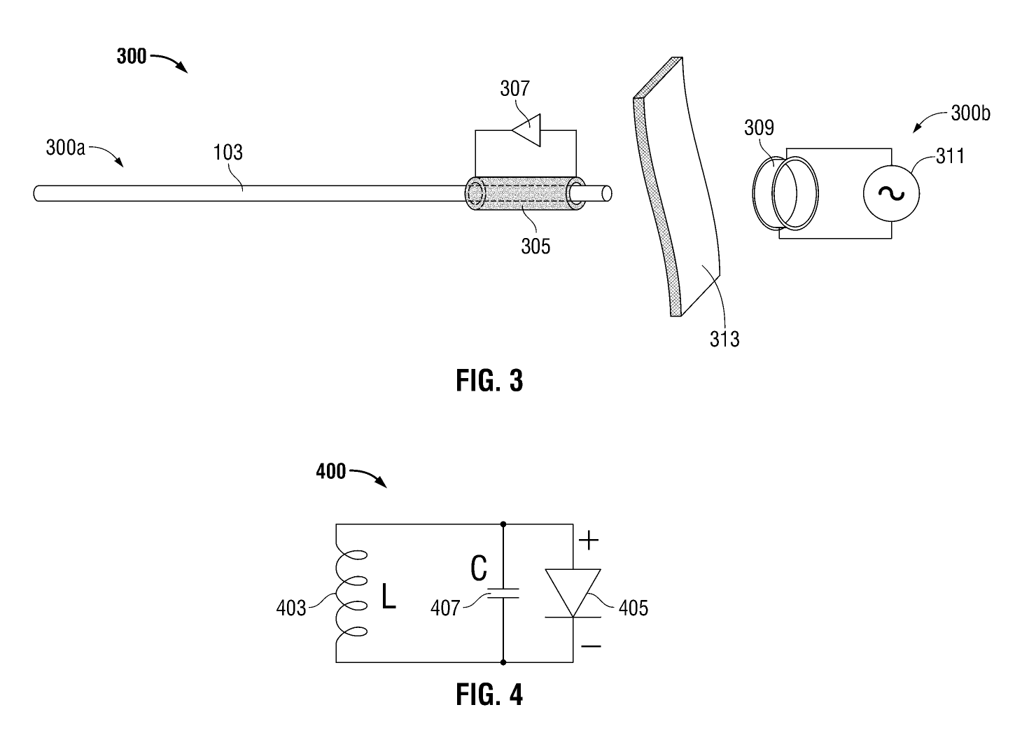

Further disclosed is a proximity sensing system for determining proximity to a desired location inside a patient. Referring to FIG. 3, the system 300 has at least one medical device 300A having an elongate member 103 including a distal portion, a proximal portion, an adapter 305, and an indicator 307. The medical device 300A, the adapter 305, and the indicator 307 may be as any other described above.

System 300 further has at least one emitter assembly 300B comprising at least one power supply 311, at least one emitter 309 operably coupled to the at least one power supply 311 and configured to selectively output electromagnetic energy. In some embodiments, the power supply 311 is an electro-surgical generator, but the generator may be any suitable source of power such as but not limited to one or more batteries or other common power sources. The power supply 311 may provide alternating current (AC) or direct current (DC).

In some embodiments, emitter 309 may comprise at least one coil that may produce a desired magnetic field when current is passed therethrough. Emitter 309 may also be an antenna configured to produce electro-magnetic radiation when current is passed therethrough.

When power supply 311 and emitter 309 are electrically coupled, a current passes through the emitter causing an electro-magnetic field to be created as a function of the current. For a coil emitter, a magnetic field is created around the coil having an intensity related to the amount of current passing through the coil. If an electromagnetic field is present, the inductor in adapter 305 can convert the EM field back into electrical current if the adapter 305 is placed within range of the EM field. The closer the adapter 305 is placed to the emitter 309, the larger the voltage drop and the stronger the current the inductor will produce. The current produced by the inductor can be directed to the indicator 307, either directly or through one or more circuit components such as but not limited to AC-to-DC converters, signal conditioning stages, and amplifiers. Furthermore, medical device 300A may have an end effector designed to manually or automatically deploy a probe, implant, or other device when the produced current reaches a certain threshold.

A method of determining the proximity of a medical device to a desired location inside a patient is also herein disclosed. The desired location may be a tissue, vessel, or organ inside the patient chosen by a clinician to accomplish a desired medical procedure such as, but not limited to minimally invasive surgery.

The method includes providing at least one power supply 311 as described above, at least one emitter 309 as described above operably coupled to the at least one power supply 311 and configured to selectively output electromagnetic energy. The method may also include providing at least one medical device 300A as described above having a elongate member 103 comprising a distal portion, a proximal portion and at least one adapter 305 as described above including at least one inductor as described above operably coupled to the distal portion and configured to inductively couple to the at least one emitter 309 and convert electromagnetic energy into electrical current. At least one indicator 307 as described above that is operably connected to the inductor may also be provided.

The method may further include placing the at least one emitter 309 outside of the patient over the desired location, as is shown in FIG. 3. Referring to FIG. 3, emitter 309 is placed on the outside of the patient near a desired location and medical device 300A is at least partially disposed within the patient and separated from emitter assembly 300B by tissue 313. For example, if a clinician is attempting to locate an organ in the abdomen of the patient, then the clinician may place the emitter 309 on the patient's abdomen approximately where the location of the organ is or should be.

The method may further include allowing electrical current to pass through the at least one emitter 309 to create an electromagnetic field. In the case where the emitter 309 is a coil, a magnetic field is created when current is passed through the coil. The method may further include positioning the distal portion of the medical device 300A at least partially inside the patient, as shown in FIG. 3. The user may enter a body cavity through an incision in the patient's tissue and position the distal portion as desired.

The method may further include receiving electromagnetic energy from the emitter 309 using the inductor in the adapter 305 thereby inductively coupling the inductor to the emitter to create electrical current in the inductor. For example, after having positioned the distal portion of the medical device inside the patient, the user may generally attempt to find the EM field being emitted by the emitter 309 in order to receive energy using the inductor.

The method may further include powering the indicator 307 using electrical current from the inductor. When the user positions the adapter 305 into the EM field created by the emitter 307, at least a portion of the received energy is converted into electrical energy for powering at least the indicator(s). In some embodiments, the indicator 307 is powered solely from the current created by the inductor, but it is possible to use power from other sources for the indicator 307.

The method may further include determining an intensity of the indicator 307 when the medical device 300A is in a first position relative to the emitter 309 and a second position relative to the emitter 309. The intensity proportionally corresponds to relative position of the adapter 305 from the emitter 309 and, thus, to the desired location. For example, if the indicator 307 includes a light, then a clinician may determine or measure the brightness or frequency of blinking as a function of location of the distal portion of the medical device 300A. If the indicator includes an audible indicator, then a clinician may determine or measure the amplitude of the sound, frequency, or tempo of beeping as a function of location of the distal portion of the medical device 300A.

The method may further include comparing the intensity at the first position with the intensity at the second position to determine if the medical device 300A has moved closer or further from the desired location. For example, if the indicator 307 includes a light, then a clinician may compare the brightness of two or more positions to determine the proximity of the distal portion of the medical device 300A to the desired location. If the indicator includes an audible indicator, then a clinician may compare the amplitude of the sound, frequency, or tempo of beeping of two or more positions to determine the proximity of the distal portion of the medical device 300A to the desired location.

When provided with a medical device as described above, a clinician may operate on a patient using the medical device as either just a locating tool or for other surgical means. When used as a locating tool, the device allows the clinician to find an in situ location to a higher level of precision which can allow for further insertion of other medical instruments at that location.

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figs. are presented only to demonstrate certain examples of the disclosure. Other elements, steps, methods, and techniques that are insubstantially different from those described above and/or in the appended claims are also intended to be within the scope of the disclosure.

What is claimed is:

1. A medical device, comprising:
an member having a proximal portion and a distal portion;
at least one adapter operably coupled to the elongate member, said adapter including at least one inductor configured to convert electromagnetic energy into an electrical current; and
at least one indicator operably coupled to said inductor.

2. The medical device of claim 1, wherein said member is an elongate member

3. The medical device of claim 1, wherein said at least one adapter is connected integrally upon said distal portion.

4. The medical device of claim 1, wherein said at least one adapter is removably connected to said distal portion.

5. The medical device of claim 1, wherein said at least one indicator is removably connected to said distal portion.

6. The medical device of claim 1, wherein said at least one indicator is connected to said proximal portion.

7. The medical device of claim 1, wherein said at least one indicator is integrally connected to said elongate member.

8. The medical device of claim 1, wherein said at least one indicator is at least one visual indicator configured to convert said electrical current into light.

9. The medical device of claim 8, wherein said indicator is a first LED.

10. The method of claim 9, wherein said first LED comprises a first color in a first condition and a second LED indicates a second color at a second condition.

11. The medical device of claim 1, wherein said at least one indicator is at least one audible indicator configured to convert said electrical current into audible sound.

12. The medical device of claim 1, wherein said adapter and indicator are configured to selectively secure to said elongate member and to be selectively removable therefrom.

13. A proximity sensing system for determining proximity to a desired location inside a patient, comprising:
at least one medical device having an elongate member including a distal portion and a proximal portion;
at least one power supply;
at least one coil operably coupled to said at least one power supply and configured to selectively output electromagnetic energy;
at least one adapter comprising at least one inductor operably coupled to said distal portion and configured to inductively couple to said coil and convert electromagnetic energy into an electrical current; and
at least one indicator operably coupled to said inductor.

14. The system of claim 13, wherein said indicator is at least one LED.

15. The system of claim 13, wherein said indicator is an LED and is connected to said distal portion.

16. A method of determining the proximity of a medical device to a desired location inside a patient, comprising;
providing at least one power supply, at least one coil operably coupled to said at least one power supply and configured to selectively output electromagnetic energy, at least one medical device having a elongate member comprising a distal portion, a proximal portion and at least one adapter including at least one inductor operably coupled to said distal portion and configured to inductively couple to said at least one coil and convert electromagnetic energy into electrical current, and at least one indicator operably connected to said inductor;
placing said at least one coil outside of said patient over said desired location;
allowing electrical current to pass through said least one coil to create electromagnetic energy;
positioning said distal portion of said medical device inside said patient;
receiving electromagnetic energy from said coil using said inductor thereby inductively coupling said inductor to said coil to create electrical current in said inductor; and
powering said indicator using electrical current from said inductor.

17. The method of claim 16, further comprising determining an intensity of said indicator when said medical device is in a first position relative to said coil and a second position relative to said coil, wherein said intensity proportionally corresponds to proximity to said coil and thus to said desired location.

18. The method of claim 17, further comprising comparing said intensity at said first position with said intensity at said second position to determine if the medical device has moved closer or further from said desired location.

19. The method of claim 16, wherein said indicator is a first LED.

20. The method of claim 18, wherein said first LED comprises a first color in a first condition and a second LED indicates a second color at a second condition.
